(19) United States
(12) Patent Application Publication
(10) Pub. No.: US 2017/0183389 A1
(43) Pub. Date: Jun. 29, 2017

(54) METHOD FOR IMPROVING PROTEIN EXPRESSION, AND COMPOSITION FOR PROTEIN EXPRESSION

(71) Applicant: ACCURNA, INC., Tokyo (JP)

(72) Inventors: Keiji ITAKA, Tokyo (JP); Kazunori KATAOKA, Tokyo (JP); Masaru IKEGAMI, Tokyo (JP); Satoshi UCHIDA, Tokyo (JP); Hirokuni UCHIDA, Tokyo (JP); Kazuya NAGATA, Tokyo (JP)

(73) Assignee: ACCURNA, INC., Tokyo (JP)

(21) Appl. No.: 15/304,900

(22) PCT Filed: Apr. 23, 2015
(86) PCT No.: PCT/JP2015/002208

§ 371 Date: Jan. 4, 2017

(30) Foreign Application Priority Data
Apr. 24, 2014 (JP) 2014-090634

(51) Int. Cl. C07K 14/65; A61K 48/00; A61K 38/30

(52) U.S. Cl. CPC C07K 14/65; A61K 38/30; A61K 48/0066

(57) ABSTRACT

The present invention provides a method for improving protein expression and a composition for protein expression. The composition is a composition for use in expressing a target protein, the composition comprising an mRNA encoding the target protein, wherein 80% or more of the mRNA molecules contained in the composition have a sequence consisting substantially of poly(A) having a length of 230 to 250 nucleotides on the 3′-end side of the protein-coding region thereof.

### CROSS-REFERENCE TO RELATED APPLICATION

This application claims the benefit of priority of Japanese Patent Application No. 2014-090634, filed Apr. 24, 2014, which is hereby incorporated by reference herein in its entirety.

### TECHNICAL FIELD

The present invention relates to a method for improving protein expression and a composition for protein expression.

### BACKGROUND ART

Proteins have extremely great potential as physiologically active substances. For example, in the treatment of a disease caused by a decrease in a specific protein, protein replacement treatment exhibits advantageous effects. Accordingly, techniques for intracellularly or extracellularly producing large amounts of proteins have been developed until now.

A protein is generated by transcription of a DNA encoding the protein into an mRNA and translation of the mRNA into the protein. In the transcription of a DNA into an mRNA, a transcription regulator is involved in and controls the production amount of the mRNA by transcriptional regulation. In contrast, the translation of an mRNA into a protein is assumed to be controlled by, for example, association of a translation initiation factor with the mRNA. It is also known that mRNAs are unstable in cells and that the stability is changed by poly(A) added to the 3′UTR of an mRNA.

Poly(A) is normally added to the 3′UTR of an mRNA by a poly(A) addition signal. Specifically, it is assumed that transcription of a DNA into an mRNA by RNA polymerase II allows a complex containing a cleavage and polyadenylation specificity factor (CPSF) to recognize the poly(A) addition signal region of 3′UTR of the mRNA to cleave the mRNA at a position 10 to 30 nucleotides downstream from the signal and thereby to start polyadenylation from the cleavage site, and that the synthesis of poly(A) is terminated when poly(A) reaches about 100 to 300 nucleotides (Non Patent Literature 1). However, the synthesis of poly(A) is not strictly controlled.

The length of poly(A) is assumed to be involved in the quantity of translation to a protein from an mRNA. For example, Non Patent Literature 2 discloses that the translation efficiency of an mRNA was enhanced when the length of poly(A) added to the 3′UTR of the mRNA was 120 nucleotides. However, synthesis of poly(A) having a controlled length is difficult, and the study has not progressed any more. At present, the actual situation is that poly(A) is enzymatically added to an mRNA based on the poly(A) addition signal region. The expression level is controlled by selecting a promoter, and various expression vectors for enhanced expression of proteins due to improvement in promoter sequence have come to the market.

### CITATION LIST

### Non Patent Literature

Non Patent Literature 1: E. Wahle, Journal of Biological Chemistry, 270: 2800-2808, 1995

Non Patent Literature 2: Holtkamp et al., Blood, 108: 4009-4017, 2006

### SUMMARY OF INVENTION

The present invention provides a method for improving protein expression and a composition for protein expression.

The present inventors have found that an mRNA having a poly(A) length in a certain range can significantly enhance its binding to eukaryotic translation initiation factor 4E (eIF4E). The present inventors have also found that an mRNA having a poly(A) length in a certain range exhibits notably high translation efficiency. The present invention is based on these findings.

The present invention provides the following aspects:

(1) A composition for use in expressing a target protein, the composition comprising:

an mRNA encoding the target protein, wherein 80% or more of the mRNA molecules contained in the composition have a sequence consisting substantially of poly(A) having a length of 230 to 250 nucleotides on the 3′-end side of the protein-coding region thereof;

(2) The composition according to above (1), wherein 90% or more of the mRNA molecules contained in the composition have a sequence consisting substantially of poly(A) having a length of 230 to 250 nucleotides on the 3′-end side of the protein-coding region thereof;

(3) The composition according to above (1), wherein 95% or more of the mRNA molecules contained in the composition have a sequence composed substantially of poly(A) having a length of 230 to 250 nucleotides on the 3′-end side of the protein-coding region thereof;

(4) The composition according to any one of above (1) to (3), wherein 20% or less of the mRNA molecules contained in the composition have poly(A) having a length of 270 or more nucleotides;

(5) A protein expression vector comprising:

a gene encoding a protein and operably linked to a promoter; and

a sequence consisting substantially of poly(A) having a length of 230 to 250 nucleotides downstream the protein-coding region of the gene encoding the protein;

(6) A method for improving an ability of an mRNA to bind to eIF4E, comprising:

adding a sequence consisting of poly(A) or consisting substantially of poly(A) to the downstream of the protein-coding region of a DNA to be transcribed into the mRNA such that the mRNA has a sequence consisting substantially of poly(A) having a length of 230 to 250 nucleotides on the 3′-end side of the protein-coding region thereof;

(7) A method for expressing a target protein in a cell in the body of a subject, comprising:

providing a composition comprising an mRNA encoding the target protein, where 80% or more of the mRNA molecules contained in the composition have a sequence consisting substantially of a poly(A) sequence having a length of 230 to 250 nucleotides on the 3′-end side of

the protein-coding region of the mRNA; and administering the composition to the subject;

(8) A pharmaceutical composition for use in treating a disease or a disorder in a subject suffering from the disease or a subject having the disorder, the pharmaceutical composition comprising an mRNA encoding a protein that can treat the disease or the disorder, wherein 80% or more of the mRNA molecules contained in the composition have a sequence composed substantially of a poly(A) sequence having a length of 230 to 250 nucleotides on the 3′-end side of the protein-coding region of the mRNA;

(9) The composition according to above (8), wherein the disease or the disorder is a disease or a disorder caused by a reduction or a lack of a protein, and the protein that can treat the disease or the disorder is the protein that is reduced or lacked in the subject;

(10) The composition according to above (8), wherein the disease or the disorder is spinal cord injury, and the protein that can treat the disease or the disorder is brain-derived neurotrophic factor (BDNF); and

(11) The composition according to above (8), wherein the disease or the disorder is peripheral nerve injury, and the protein that can treat the disease or the disorder is insulin-like growth factor (IGF-1).

### BRIEF DESCRIPTION OF DRAWINGS

FIG. 1 is an electrophoresis photograph of purified mRNAs.

FIG. 2 includes graphs showing the effects of poly(A) length on the quantity of binding of an mRNA to the proteins shown in the graphs.

FIG. 3 is a graph showing a relationship between the poly(A) length and the translation efficiency in cultured cells.

FIG. 4 includes graphs showing relationships between the poly(A) length and the translation efficiency in cell-free extract systems, where FIG. 4A shows the results in a human cell-free extract system, and FIG. 4B shows the results in a rabbit reticulocyte lysate.

FIG. 5 is a graph showing a relationship between the poly(A) length and the translation efficiency in vivo.

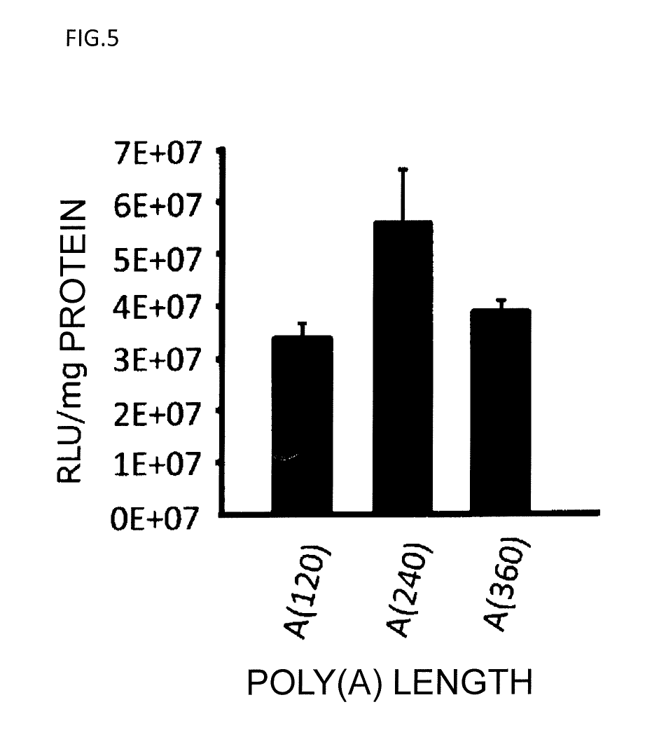

FIG. 6 includes diagrams showing the influence of poly(A) length on the therapeutic effect in a sciatic nerve damage model.

### DESCRIPTION OF EMBODIMENTS

In the present invention, the term “mRNA” refers to messenger RNA. The mRNA is preferably derived from a eukaryote. Examples of the eukaryote include bacteria, such as Escherichia coli; fungi, such as yeast; insects, such as silkworm; and mammals, such as human. The eukaryote is more preferably Escherichia coli, yeast, silkworm, or human. In a living organism, an mRNA is generated by transcription from a DNA. After the completion of the transcription, poly(A) is added to the 3′-end of the mRNA. In the mRNA, 3′ untranslated region (3′UTR) normally lies between the protein-coding region (CDS) and the poly(A).

As used herein, the term “protein-coding region” refers to a region, on a DNA, encoding a protein or a region, on an RNA, encoding a protein. The term “3′-end side of the protein-coding region” refers to, in an mRNA, a region outside the protein-coding region and lying on the 3′-end side and preferably refer to a region further outside the 3′UTR, and may be meant to the 3′-end of the 3′UTR.

As used herein, the term “upstream” refers to a region lying on the opposite side of the direction of reading genetic information when viewed from a protein-coding region of a gene. As used herein, the term “downstream” refers to a region lying in the direction of reading genetic information when viewed from a protein-coding region of a gene.

Herein, the term “poly(A)” refers to a DNA or an RNA made by polymerization of adenine. Herein, the term “sequence consisting substantially of poly(A)” represents that 90% or more, preferably 95% or more, more preferably 97% or more, and most preferably 100% of all nucleotides of the sequence consist of adenine (A). That is, a sequence consisting substantially of poly(A) may contain nucleotides other than adenine (A). In a sequence consisting substantially of poly(A), the nucleotides other than adenine (A) is concentrated in, for example, three or less, preferably two or less, and more preferably one region. In a sequence consisting substantially of poly(A), the nucleotides other than adenine (A) are, for example, restriction enzyme cleavage sites.

Herein, the term “promoter” refers to a transcriptional regulatory region for an mRNA lying on a DNA. In the present invention, in vitro transcriptional promoters and eukaryotic promoters can be used as the “promoter”. Examples of the in vitro transcriptional promoter include SP6 promoter, T3 promoter, and T7 promoter, and these promoters can be used in the present invention. Examples of the eukaryotic promoter include promoters of bacteria such as Escherichia coli and promoters of mammals, and promoters appropriately selected depending on the host cell can be used in the present invention. Examples of the promoter for Escherichia coli include T7 promoter, tac promoter, and T7 lac promoter. Examples of the promoter for mammals include CMV promoter; β actin promoters, such as CAG promoter; EF1α promoter; and SRα promoter. Herein, the term “terminator” refers to the termination signal of transcription.

A process of polyadenylation in general translation of an mRNA in vivo can be described as follows. It is assumed that transcription of a DNA into an mRNA by RNA polymerase II allows a complex containing a cleavage and polyadenylation specificity factor (CPSF) to recognize the poly(A) addition signal region of 3′UTR of the mRNA to cleave the mRNA downstream the signal and thereby to start polyadenylation from the cleavage site.

A poly(A) tail functions as a binding site for a poly(A)-binding protein and accelerates the transport of the mRNA to the outside of the nucleus. The poly(A) tail also has a role of protecting the mRNAs from degradation in cytoplasm. Consequently, the poly(A) tail contributes to an improvement in efficiency of translation into a protein.

eIF4E, also called Eukaryotic translation initiation factor 4E, has important roles in the translational control of an mRNA, involving in recognition of the 5′ cap structure of the mRNA and playing a role in circularization of the mRNA through eIF4G and poly(A)-binding protein (PABP).

The present inventors have found that although the strength of binding of an mRNA having long poly(A) to PABP increases, the formation of a complex of the mRNA and eIF4E is significantly enhanced at a specific poly(A) length (specifically, 240 nucleotides). In addition, if the poly(A) length is longer than a certain value, the strength of binding to PABP is increased, but the strength of binding to eIF4E is decreased. Since it is assumed that eIF4E binds to (or forms a complex with) the poly(A) of an mRNA through PABP, the result that an mRNA having long poly(A), which strengthens the binding to PABP, weakens the binding to eIF4E is very surprising.

In addition, according to the present invention, when the poly(A) length of an mRNA is 210 or less nucleotides or 270 or more nucleotides, the translation efficiency is significantly decreased compared with a case of 240 nucleotides. In the present invention, therefore, the poly(A) length of an mRNA is preferably controlled to a certain length. The term “a certain length” herein is, for example, 220 to 260 nucleotides, preferably 225 to 255 nucleotides, particularly preferably 230 to 250 nucleotides, further preferably 230 to 245 nucleotides, and most preferably 235 to 245 nucleotides. In a composition of a certain embodiment, mRNAs having poly(A) of 270 or more nucleotides are reduced in their amounts or are removed.

The poly(A) length can be easily controlled by removing the poly(A) addition signal. Accordingly, in a certain embodiment of the present invention, the mRNA does not have a poly(A) addition signal. In another embodiment of the present invention, the DNA that is transcribed into an mRNA does not have a poly(A) addition signal between the protein-coding region and the terminator. In a certain another embodiment, the DNA that is transcribed into an mRNA does not have a poly(A) addition signal between the protein-coding region and the terminator, but instead has a sequence consisting substantially of poly(A) having a certain length.

The present inventors have revealed that an mRNA including poly(A) having a certain length has improved ability to bind to eIF4E. Accordingly, an aspect of the present invention provides a method for improving the ability of an mRNA to bind to eIF4E, wherein the method comprises adding a sequence consisting of poly(A) or consisting substantially of poly(A) to the downstream side of the protein-coding region of a DNA to be transcribed into an mRNA such that the mRNA has a sequence consisting substantially of poly(A) having a certain length on the 3′-end side of the protein-coding region there. In a preferred embodiment, the certain length is of 230 to 250 nucleotides.

An mRNA is given by transcription of the region between the transcription start region and the terminator on a DNA by RNA polymerase II. Accordingly, a sequence consisting of poly(A) or consisting substantially of poly(A) can be inserted into between the protein-coding region and the terminator of a DNA.

The method for improving the ability of an mRNA to bind to eIF4E according to the present invention may further comprise removing of the poly(A) addition signal. The poly(A) addition signal is typically AATAAA (or AAUAAA), and those skilled in the art can readily remove the poly(A) addition signal. The poly(A) addition signal is contained in the region between the CDS and the terminator or in the 3′UTR and therefore can be removed also by removing a part or whole of these regions.

In a certain embodiment, the method for improving the ability of an mRNA to bind to eIF4E according to the present invention may further comprise reducing or removing mRNAs including poly(A) having a length of 270 or more nucleotides. In a certain embodiment of the present invention, the method may further comprise reducing or removing mRNAs including poly(A) having a length of 210 or less nucleotides. In a certain embodiment of the present invention, the method may further comprise reducing or removing mRNAs including poly(A) having a length of 210 or less nucleotides and a length of 270 or more nucleotides.

The present inventors have revealed that the translation efficiency is significantly improved when the mRNA has a poly(A) length of 240 nucleotides. The translation efficiency was notably higher than the efficiencies of an mRNA having a poly(A) length of 210 nucleotides and an mRNA having a poly(A) length of 270 nucleotides. Since it has been conventionally assumed that the stability increases and the translation efficiency is enhanced with the length of poly(A), it was unexpected that the translation efficiency of an mRNA increases within a specific and significantly narrow range of the poly(A) length and that the increase is notable.

Accordingly, an aspect of the present invention provides a composition for use in expressing a target protein, comprising an mRNA encoding a target protein, and 80% or more (molecular number rate, the same applies hereinafter), preferably 90% or more, more preferably 95% or more, further preferably 97% or more, further more preferably 99% or more, and most preferably 100% of the mRNA molecules contained in the composition have a sequence consisting substantially of poly(A) having a certain length (for example, 230 to 250 nucleotides) on the 3′-end side of the protein-coding region thereof. As described above, the poly(A) length of the mRNA is preferably controlled to a certain length. The molecular number rate (%) of an mRNA can be determined by a method well known to those skilled in the art. The molecular number rate can be determined, for example, from an electrophoresis pattern prepared by electrophoresis of the mRNA encoding the target protein. In addition, electrophoresis showing excellent quantitativeness by an RNA analysis microchip has been developed and can be used for calculation of the molecular number rate of an mRNA.

In a certain embodiment, the composition of the present invention contains mRNA molecules encoding the target protein contained in the composition and including poly(A) having a length of 270 or more nucleotides in an amount of 20% or less, preferably 15% or less, more preferably 10% or less, further preferably 5% or less, further more preferably 3% or less, and particularly preferably 1% or less, and most preferably, the composition does substantially not contain mRNA molecules encoding the target protein and including poly(A) having a length of 270 or more nucleotides. In a certain embodiment, the composition of the present invention contains mRNA molecules encoding the target protein and including poly(A) having a length of 210 or less nucleotides in an amount of 20% or less, preferably 15% or less, more preferably 10% or less, further preferably 5% or less, further more preferably 3% or less, and particularly preferably 1% or less, and most preferably, the composition does substantially not contain mRNA molecules encoding the target protein and including poly(A) having a length of 210 or less nucleotides. In a certain embodiment of the present invention, the composition contains the mRNA molecules encoding the target protein and including poly(A) having a length of 210 or less nucleotides and a length of 270 or more nucleotides in an amount of 20% or less, preferably 15% or less, more preferably 10% or less, further preferably 5% or less, further more preferably 3% or less, and particularly preferably 1% or less, and most preferably, the composition does substantially not contain mRNA molecules including poly(A) having a length of 210 or less nucleotides or 270 or more nucleotides.

In naturally occurring mRNAs or mRNAs occurring by addition of poly(A) by a poly(A) addition signal, the length of poly(A) distributes in a broad range. In contrast, in the composition of the present invention, 80% or more of the mRNA molecules encoding a target protein contained in the composition have a certain poly(A) length (for example, 230 to 250 nucleotides). If the poly(A) has a certain length (for example, 230 to 250 nucleotides), the translation efficiency is significantly increased, which is extremely advantageous from the viewpoint of translation efficiency. In a preferred embodiment of the present invention, 90% or more, further preferably 95% or more, further more preferably 97% or more, particularly preferably 99% or more, and most preferably 100% of the mRNA molecules encoding a target protein contained in the composition have a sequence consisting substantially of poly(A) having a certain length (for example, 230 to 250 nucleotides) on the 3′-end side of the protein-coding region thereof. In a certain embodiment of the present invention, the mRNA does not have a poly(A) addition signal.

The composition of the present invention may contain one kind of mRNA or may be a mixture of a plurality of kinds of mRNAs. The composition of the present invention may be a mixture of mRNAs having a variety of lengths of poly(A). The composition of the present invention may contain a carrier, such as a buffer solution, or an excipient, in addition to the mRNA. The composition of the present invention may contain a carrier for mRNA delivery.

The composition of the present invention can be used for expressing a protein in vitro or in in vivo cells. The composition of the present invention can also be used for expressing a protein in an in vitro translation system, such as a cell-free extract system.

An aspect of the present invention provides a protein expression vector including a gene encoding a protein and operably linked to a promoter and including a sequence consisting substantially of poly(A) having a certain length (for example, 230 to 250 nucleotides) downstream the protein-coding region of the gene encoding the protein. The protein expression vector of the present invention is used in production of an mRNA to which a sequence consisting substantially of poly(A) having a certain length (for example, 230 to 250 nucleotides) in the 3′UTR region is added. In a certain embodiment of the present invention, no poly(A) addition signal lies between the CDS and the terminator.

An aspect of the present invention provides a method for expressing a target protein in a cell in the body of a subject, comprising providing a composition containing an mRNA encoding the target protein, where 80% or more, preferably 90% or more, more preferably 95% or more, further preferably 97% or more, further more preferably 99% or more, and most preferably 100% of the mRNA molecules contained in the composition have a sequence consisting substantially of a poly(A) sequence having a certain length (for example, 230 to 250 nucleotides) on the 3′-end side of the protein-coding region of the mRNA; and administering the composition to the subject.

In a certain embodiment, the method for expressing a target protein in a cell in the body of a subject according to the present invention may further comprise reducing or removing mRNA molecules including poly(A) having a length of 270 or more nucleotides. In a certain embodiment of the present invention, the method may further comprise reducing or removing mRNA molecules including poly(A) having a length of 210 or less nucleotides. In a certain embodiment of the present invention, the method may further comprise reducing or removing mRNA molecules including poly(A) having a length of 210 or less nucleotides and 270 or more nucleotides.

Another aspect of the present invention provides a method for treating a disease or a disorder in a subject suffering from the disease or a subject having the disorder, comprising administering to the subject a composition containing an mRNA encoding a protein that can treat the disease or the disorder, where 80% or more, preferably 90% or more, more preferably 95% or more, further preferably 97% or more, further more preferably 99% or more, and most preferably 100% of the mRNA molecules contained in the composition have a sequence consisting substantially of a poly(A) sequence having a certain length (for example, 230 to 250 nucleotides) on the 3′-end side of the protein-coding region of the mRNA.

In a specific embodiment of the method for treating a disease or a disorder according to the present invention, the disease or the disorder is peripheral nerve injury, and the protein that can treat the disease or the disorder is insulin-like growth factor (IGF-1). In a specific embodiment of the present invention, the peripheral nerve injury can be a sciatic nerve damage. IGF-1 exhibits a muscle hypertrophy effect (or muscle atrophy preventing effect) by, for example, intramuscular administration of an mRNA encoding IGF-1, has a nerve regeneration enhancing action, accelerates regeneration of damaged nerve, and can recover the motor function of the subject.

In another specific embodiment of the method for treating a disease or a disorder according to the present invention, the disease or the disorder is a spinal cord injury, and the protein that can treat the disease or the disorder is brain-derived neurotrophic factor (BDNF). BDNF enhances the recovery of nervous function after the spinal cord injury by, for example, intrathecal administration of an mRNA encoding BDNF.

In another specific embodiment of the method for treating a disease or a disorder of the present invention, the disease or the disorder is a disease or a disorder caused by a reduction or a lack of a protein, and the protein that can treat the disease or the disorder is the protein that is reduced or lacked in the disease or the disorder. That is, this specific embodiment is protein replacement treatment. In another specific embodiment of the method for treating a disease or a disorder of the present invention, the protein that can treat the disease or the disorder is a secretory factor or an accelerator accelerating the production of a secretory factor, and the disease or the disorder is a disease or a disorder caused by a reduction or a lack of the secretory factor.

Another aspect of the present invention provides a pharmaceutical composition for use in the method for treating a disease or a disorder according to the present invention. That is, the present invention provides a pharmaceutical composition for use in treating a disease or a disorder in a subject suffering from the disease or a subject having the disorder, comprising an mRNA encoding a protein that can treat the disease or the disorder, where 80% or more, preferably 90% or more, more preferably 95% or more, further preferably 97% or more, further more preferably 99% or more, and most preferably 100% of the mRNA molecules contained in the composition have a sequence consisting substantially of a poly(A) sequence having a certain length (for example, 230 to 250 nucleotides) on the 3′-end side of the protein-coding region of the mRNA. In a specific embodiment of the pharmaceutical composition of the present invention, the disease or the disorder is peripheral nerve injury, and the protein that can treat the disease or the disorder is insulin-like growth factor (IGF-1). In a specific embodiment of the present invention, the peripheral nerve injury can be a sciatic nerve damage. In another specific embodiment of the pharmaceutical composition of the present invention, the disease or the disorder is spinal cord injury, and the protein that can treat the disease or the disorder is brain-derived neurotrophic factor (BDNF).

Another embodiment of the pharmaceutical composition of the present invention provides a pharmaceutical composition for treating and/or preventing a disease or a disorder caused by a reduction or a lack of a protein, comprising an mRNA encoding the protein, where 80% or more, preferably 90% or more, more preferably 95% or more, further preferably 97% or more, further more preferably 99% or more, and most preferably 100% of the mRNA molecules contained in the composition have a sequence consisting substantially of a poly(A) sequence having a certain length (for example, 230 to 250 nucleotides) on the 3′-end side of the protein-coding region of the mRNA. In a specific embodiment, in the pharmaceutical composition of the present invention, the protein is a secretory factor.

Another aspect of the present invention relates to a use of an mRNA in production of a pharmaceutical composition for use in treating a disease or a disorder in a subject suffering from the disease or a subject having the disorder, wherein the mRNA encodes a protein that can treat the disease or the disorder and have a sequence consisting substantially of a poly(A) sequence having a certain length (for example, 230 to 250 nucleotides) on the 3′-end side of the protein-coding region of the mRNA. A specific embodiment of the present invention provides a use of a composition in production of a pharmaceutical composition for use in treating a disease or a disorder in a subject suffering from the disease or a subject having the disorder, wherein the composition comprises an mRNA encoding a protein that can treat the disease or the disorder, and wherein 80% or more, preferably 90% or more, more preferably 95% or more, further preferably 97% or more, further more preferably 99% or more, and most preferably 100% of the mRNA molecules contained in the composition have a sequence consisting substantially of a poly(A) sequence having a certain length (for example, 230 to 250 nucleotides) on the 3′-end side of the protein-coding region of the mRNA. In a specific embodiment of the use of the present invention, the disease or the disorder is peripheral nerve injury, and the protein that can treat the disease or the disorder is insulin-like growth factor (IGF-1). In a specific embodiment of the present invention, the peripheral nerve injury can be a sciatic nerve damage. In another specific embodiment of the use of the present invention, the disease or the disorder is spinal cord injury, and the protein that can treat the disease or the disorder is brain-derived neurotrophic factor (BDNF). In another embodiment of the use of the present invention, the disease or the disorder is a disease or a disorder caused by a reduction or a lack of a protein, and the protein that can treat the disease or the disorder is the protein that is reduced or lacked in the disease or the disorder. In a specific embodiment of the use of the present invention, the protein is a secretory factor.

The pharmaceutical composition of the present invention may contain one kind of mRNA or may be a mixture of a plurality of kinds of mRNAs. The pharmaceutical composition of the present invention may be a mixture of mRNAs having a variety of lengths of poly(A). The pharmaceutical composition of the present invention may contain a pharmaceutically acceptable carrier or excipient, such as a buffer solution in addition to the mRNA. The pharmaceutical composition of the present invention may contain a carrier for mRNA delivery. The pharmaceutical composition of the present invention can be administered, for example, by, but not be particularly limited to, parenteral administration. Examples of the parenteral administration include, but not be limited to, intramuscular administration, intraventricular administration, intravenous administration, intraperitoneal administration, intracerebroventricular administration, intraocular administration, subcutaneous administration, intranasal administration, intravaginal administration, and intrathecal administration. By such administration, the pharmaceutical composition can be administered in the present invention. In a certain embodiment, the pharmaceutical composition of the present invention is provided as an injection. The pharmaceutical composition of the present invention can be administered systemically or locally by an appropriate dosage form depending on the disease or the disorder.

As used herein, the term “treat” is meant to induce cure, prevention, or remission of a disease or a disorder or a reduction in rate of progress of a disease or a disorder. The treatment can be achieved by administering a therapeutically effective amount of the pharmaceutical composition.

As used herein, the term “subject” is preferably a human subject or a human patient.

In the composition or the pharmaceutical composition of the present invention, in a certain embodiment, the mRNA forms a polyion complex with PEG-PAsp(DET) in the composition. Herein, the term “PEG-PAsp(DET)” refers to a copolymer of a poly(ethylene glycol) block and a polyaspartic acid derivative block. In the PEG-PAsp(DET) used in the present invention, although the PEG has an average degree of polymerization of 5 to 20000, preferably 10 to 5000, and more preferably 40 to 500, the degree of polymerization of the PEG is not limited as long as the formation of the polyion complex of the block copolymer and mRNA is not blocked. In the PEG-PAsp(DET) used in the present invention, in a certain embodiment, the aspartic acid derivative is aspartic acid of which the carboxyl group in the side chain is substituted with a diethyltriamine (DET) group (—NH—CH₂—CH₂—NH—CH₂—CH₂—NH₂). The structure of poly(Asp(DET)) is represented by the following chemical formula:

### Poly(Asp(DET))

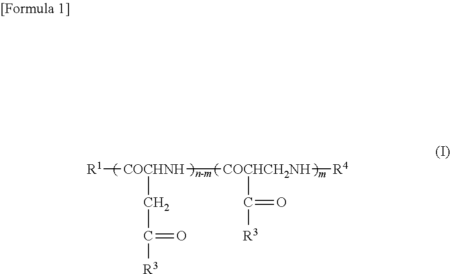

{where

R¹ represents a hydroxy group, a protecting group, a hydrophobic group, or a polymerizable group;

R⁴ represents H, a protecting group, a hydrophobic group, or a polymerizable group;

R³ represents a group represented by —(NH—(CH₂)₂)₂—NH₂;

n represents an integer of 0 to 5000, for example, an integer of 0 to 500;

m represents an integer of 0 to 5000, for example, an integer of 0 to 500;

m+n represents an integer of 2 to 5000, for example, an integer of 2 to 500; and

n−m represents an integer of 0 or more, and

in the formula, although the repeating units are shown in a specific order for convenience of description, the repeating units can be present in no particular order, and the repeating units may be present in random order and may be the same or different,

provided that when a polycation block forms a copolymer with poly(ethylene glycol), R¹ or R⁴ represents a bond, and poly(ethylene glycol) forms a copolymer with a polycation block through the bond}. Additionally, in the polymer represented by Formula (I), the repeating units are bonded by a peptide bond.

### EXAMPLES

### Example 1: Construction of Protein Expression Plasmid

In this example, plasmids having poly(A) sequences having different lengths downstream the protein-coding regions of mRNAs were constructed.

GLuc gene (derived animal species Gaussia princeps) and NLuc gene (derived animal species Oplophorus gracilirostris) were used as luciferase genes, and were cut out from pCMV-GLuc Control Plasmid (New England Biolabs Inc., Catalog No. N8081S) and pNL1.1[NLuc] Vector (Promega Corporation, Catalog No. N1001), respectively, with restriction enzymes HindIII and XbaI. Each luciferase gene was cloned between HindIII and XbaI cleavage sites in a multicloning site under the control of T7 promoter of pSP73 vector (Promega Corporation, Catalog No. P2221). The resulting plasmid is called pSP73-Luc plasmid.

A poly(A) sequence (having a length of 120, 180, 210, 240, 270, or 360 nucleotides) was then inserted into the downstream of the luciferase gene by in-frame connection. The poly(A) tail sequence was produced using an oligo DNA, specifically, as follows.

### 1-1. Production of Plasmid Including Poly(A) Having a Chain Length of 120 Nucleotides

Two oligo DNAs 5′-AATTC-A₁₂₁-GAGACGA-3′ and 5′-GATCTCGTCTC-T₁₂₁-G-3′ were annealed to produce a double-stranded DNA, and the double-stranded DNA was inserted into pSP73-Luc plasmid linearized using restriction enzymes EcoRI and BglII to produce a plasmid including poly(A) having a chain length of 120 nucleotides (A(120)). In the above-mentioned sequence, Aₙ represents that adenines are continuously present to form a length of n nucleotides. For example, A₁₂₁ means that adenines continue to form a length of 121 nucleotides.

### 1-2. Production of Plasmids Including Poly(A) Having a Chain Length of 180, 210, or 240 Nucleotides

A double-stranded DNA produced from two oligo DNAs 5′-AATTC-A₁₂₀-GATATCA-3′ and 5′-GATCTGATATC-T₁₂₀-G-3′ was inserted into pSP73-Luc plasmid linearized with restriction enzymes EcoRI and BglII to produce pSP73-GLuc-A(120)-EcoRV plasmid including poly(A) having a length of 120 nucleotides and a restriction enzyme EcoRV recognition sequence. The resulting plasmid was then linearized with restriction enzymes EcoRV and BglII. Subsequently, a double-stranded DNA produced from two oligo DNAs 5′-G-Aₓ-GAGACGA-3′ and 5′-GATCTCGTCTC-Tₓ-C-3′ (herein, x represents 61, 91, or 121) was inserted into the restriction enzyme site of the resulting plasmid to produce plasmids including poly(A) having a chain length of 180, 210, or 240 nucleotides.

### 1-3. Production of Plasmid Including Poly(A) Having a Chain Length of 270 or 360 Nucleotides

A double-stranded DNA produced from two oligo DNAs 5′-G-A₁₂₀-GATATCA-3′ and 5′-GATCTGATATC-T₁₂₀-C-3′ was inserted into pSP73-Luc-A(120)-EcoRV plasmid linearized using restriction enzymes EcoRV and BglII to produce pSP73-GLuc-A(240)-EcoRV plasmid. The produced plasmid was linearized using restriction enzymes EcoRV and BglII. Subsequently, a double-stranded DNA produced from two oligo DNAs 5′-G-Aₓ-GAGACGA-3′ and 5′-GATCTCGTCTC-Tₓ-C-3′ (herein, x represents 31 or 121) was inserted into the restriction enzyme site of the resulting plasmid to produce plasmids including poly(A) having a chain length of 270 or 360 nucleotides.

Hereinafter, the resulting plasmid is expressed as “pSP73-Luc-A (nucleotide length of poly(A))”. For example, a poly(A) sequence having a length of 120 nucleotides is expressed as pSP73-Luc-A(120) in the specification.

The protein expression plasmid was replicated in Escherichia coli and was then purified to be used in the subsequent examples by an ordinary method in the art.

### Example 2: Ability of mRNA to Bind to Protein

In this example, the ability of an mRNA to bind to a protein was verified.

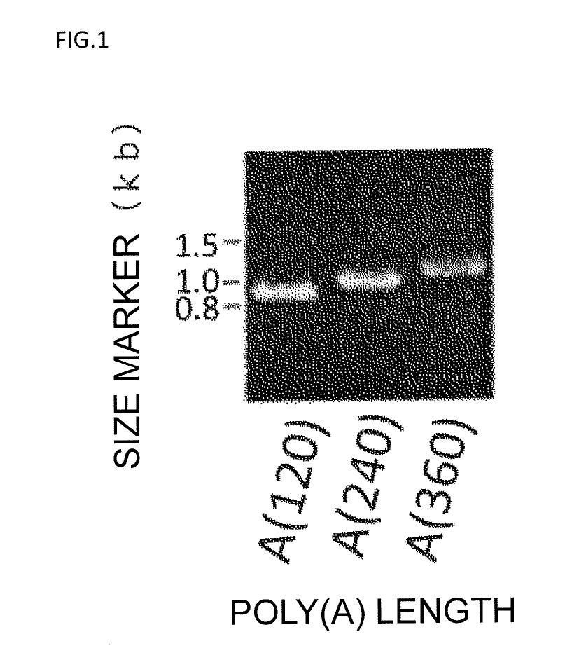

The plasmids including poly(A) having a chain length of 120, 180, or 210 nucleotides were converted to linearized DNAs with type IIS restriction enzyme BsmBI, and the linearized DNAs were purified by agarose electrophoresis. The plasmids including poly(A) having a chain length of 240, 270, or 360 nucleotides were converted to linearized DNAs with two kinds of restriction enzymes BsmBI and HpaI, and the linearized DNAs were purified by agarose electrophoresis. The purified linearized DNAs had a luciferease gene under the control of T7 promoter. The resulting linearized DNAs were in vitro transcribed using mMESSAGE mMACHINE T7Ultra Kit (Ambion, Inc.) according to the manufacturer's manual to prepare mRNAs. The resulting mRNAs were purified with RNeasy Mini Kit (Qiagen N.V.). The results of agarose electrophoresis of the purified mRNAs are shown in FIG. 1. FIG. 1 shows the results of electrophoresis of mRNAs including A(120), A(240), and A(360).

The mRNA bound to an intranuclear protein was prepared as follows: Huh7 cells were seeded in a 96-well plate at a density of 5000 cells/well and were cultured for 24 hours. The culture medium used was a 10% FBS-containing DMEM. The GLuc mRNA including poly(A) having any of various lengths purified in Example 1 was added to each well at an amount of 190 ng to be introduced into the Huh7 cells with a gene introduction reagent Lipofectamine LTX (Life technologies, Inc.). After 24 hours from the introduction, cell lysate samples were prepared using Dynabeads Co-Immunoprecipitation Kit (Life technologies, Inc.).

The binding between an mRNA and a protein was verified by immunoprecipitation. That is, the mRNA formed a complex with a protein, PABP or eIF4E, was precipitated using an antibody against PABP or eIF4E, and the amount of the precipitated mRNA was used as an index of the binding of the mRNA to the protein. Specifically, the antibody used was an anti-PABP antibody (Abcam plc., Catalog No. ab21060) or an anti-eIF4E antibody (Santa Cruz Biotechnology, Inc., Catalog No. sc-13963). The immunoprecipitation was performed using Dynabeads Co-Immunoprecipitation Kit (Life technologies, Inc.) by incubation at 4° C. for 30 minutes to bind the antibody to the protein. Subsequently, the total RNAs were collected with RNeasy Mini Kit (QIAGEN N.V.), and cDNAs were produced from the mRNAs contained in the total RNAs with RevertraAce qPCR RT Master Mix with gDNA Remover (TOYOBO Co., Ltd.).

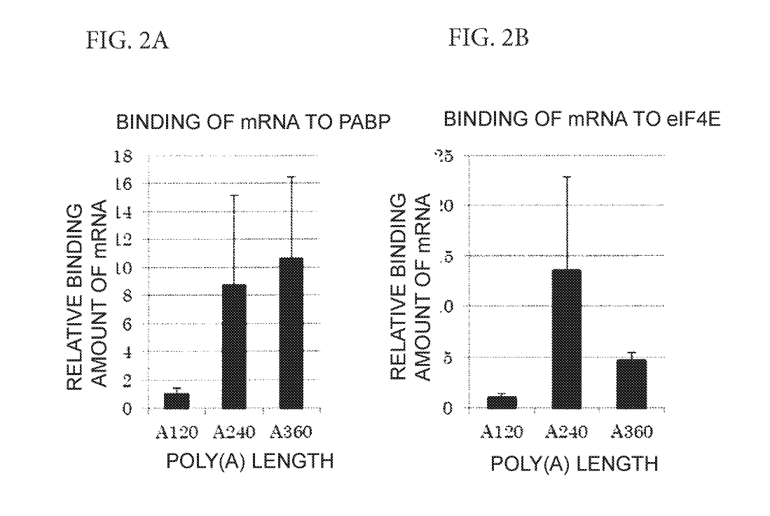

The amount of the GLuc mRNA bound to the protein and precipitated with the antibody was quantitatively measured by a quantitative polymerase chain reaction (quantitative PCR) using ABI Prism 7500 Sequence Detector (Applied Biosystems). The primers used were forward primer: 5′-TTGAACCCAGGAATCTCAGG-3′ and reverse primer: 5′-CACGCCCAAGATGAAGAAGT-3′. The results of the quantitative measurement were then standardized relative to the amount, which was defined as 1, of the mRNA having a poly(A) length of 120. The results were as shown in FIG. 2.

The results of the binding of mRNAs to PABP will be described by FIG. 2A. As shown in FIG. 2A, the mRNA including A(240) (i.e., the poly(A) length was 240 nucleotides) significantly bound to PABP compared with the mRNA including A(120) (i.e., the poly(A) length was 120 nucleotides). The mRNA including A(360) bound to a larger amount of PABP compared with the mRNA including A(240). This demonstrated that an mRNA including long poly(A) length can advantageously bind to PABP.

The results of the binding of mRNAs to eIF4E will be then described by FIG. 2B. As shown in FIG. 2B, it was shown that the mRNA including A(240) bound to a significantly larger amount of eIF4E compared with the mRNAs including A(120) or A(360). It was revealed that poly(A) having a specific length is suitable for binding to eIF4E, and it was suggested that poly(A) being too long has a risk of blocking the binding to eIF4E.

Since PABP is known to bind to the poly(A) of an mRNA, the result that PABP advantageously binds to an mRNA including long poly(A) is an expected result. Since eIF4E is assumed to also bind to the poly(A) of an mRNA through PABP, it has been similarly expected that eIF4E advantageously binds to an mRNA including long poly(A). However, the results mentioned above demonstrated that eIF4E strongly binds to an mRNA including poly(A) having a specific length (specifically, an mRNA including A(240)) and significantly strongly binds to the mRNA compared with mRNAs including A(120) or A(360). That is, although an mRNA including A(360) strongly binds to PABP, the binding to eIF4E was surprisingly weakened.

### Example 3: Efficiency of In Vitro Translation into Protein from mRNA

In this example, the efficiency of translation of the resulting mRNA into a protein was investigated.

Huh7 cells were seeded in a 96-well plate at a density of 5000 cells/well and were cultured for 24 hours. The culture medium used was a 10% FBS-containing DMEM. The GLuc mRNA including poly(A) having any of various lengths purified in Example 1 was introduced at an amount of 190 ng into the Huh7 cells with a gene introduction reagent Lipofectamine LTX (Life technologies, Inc.). In addition, as a control, an mRNA to which poly(A) was enzymatically added using a poly(A) addition signal was prepared. The pSP73-Luc plasmid prepared in Example 1 was cleaved using a restriction enzyme NdeI. The linearized plasmid was in vitro translated using mMESSAGE mMACHINE T7 Ultra Kit (Ambion, Inc.) according to the manufacturer's manual to produce an mRNA. Subsequently, according to the same manufacture's manual, poly(A) was added to the resulting mRNA (reaction conditions: 37° C., 45 min). The resulting mRNA was purified with RNeasy Mini Kit (Qiagen N.V.) and was introduced into the Huh7 cells as described above.

The quantitative measurement was performed based on the amount of luminescence from the luciferase.

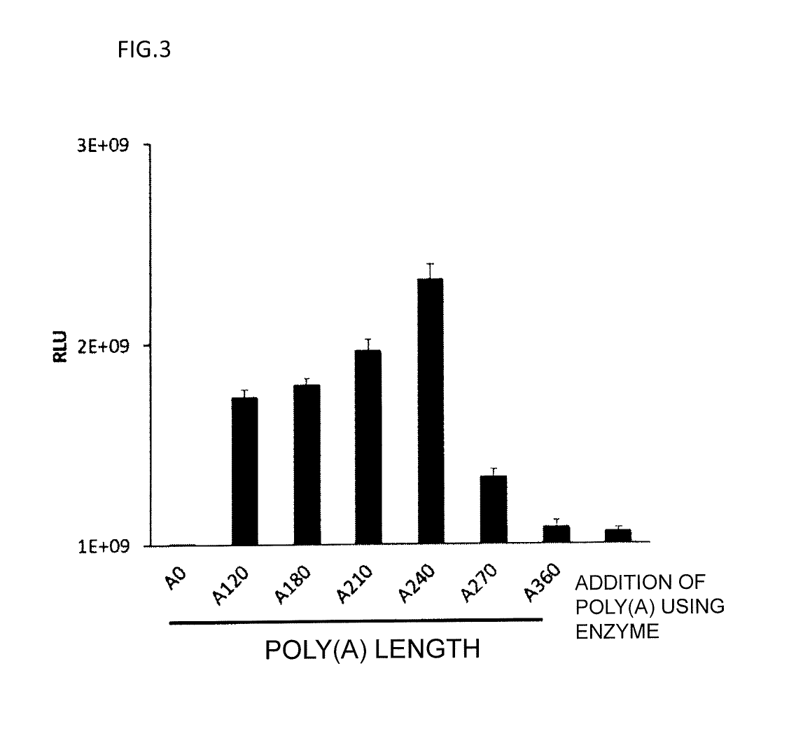

Specifically, the amount of luciferase luminescence from each mRNA was measured using Renilla Luciferase, assay System (Promega Corporation) with GloMax™ 96 microplate luminometer (Promega Corporation). The amount of luminescence was determined by relative luciferase units (RLU). The results were as shown in FIG. 3.

As shown in FIG. 3, the mRNAs having A(120), A(180), or A(210) showed approximately the same degree of protein expression. Compared with these expression levels, the mRNA having A(240) showed a significantly high level of protein expression. This increase in expression level was statistically significant. In contrast, the protein expression of the mRNAs having A(270) or A(360) was statistically significantly lower compared with that of the mRNA having A(240). These results revealed that the protein expression when the poly(A) length is 240 is significantly increased compared with those when the poly(A) length is 210 or 270. The significant decrease in the translation efficiency of the mRNA having A(270) suggested that poly(A) of 270 or more nucleotides gained an activity of blocking the translation.

The mRNA having A(90) showed protein expression equivalent to the level of the mRNA having A(120) (data not shown). In addition, when luciferase is expressed using a protein expression plasmid including a poly(A) addition signal (AATAAA) (the above-mentioned control) instead of poly(A), the RLU was only the same as that of the mRNA having A(360). That is, the expression level of a target protein by the mRNA having A(240) was much higher than the expression level by an mRNA to which poly(A) was enzymatically added, which is similar to a natural mRNA.

A(180), A(210), A(240), A(270), and A(360) each contain an intervening sequence (GAUG sequence) derived from a restriction enzyme site between poly(A) and poly(A). In order verify the influence of GAUG on protein expression, A(120) and A(60)-GAUG-A(60) were produced as in above, and the expression levels were compared. The expression level of A(60)-GAUG-A(60) was about 80% of that of A(120), which revealed that the influence of the intervening sequence is limited (data not shown).

The same results were observed in cell-free translation systems (rabbit reticulocyte lysate and human cell-free extract). Specifically, Rabbit Reticulocyte Lysate, Untreated (Promega Corporation) was used as the rabbit reticulocyte lysate, and Human Cell-Free Protein Expression System (Takara Bio Inc.) was used as the human cell-free extract. GLuc mRNAs having each poly(A) length were incubated at 30° C. for 120 minutes or at 32° C. for 30 minutes. The expression levels were quantitatively measured based on the amounts of luminescence from luciferase. The amount of luciferase luminescence from each mRNA was measured using Renilla Luciferase assay System (Promega Corporation) with GloMax™ 96 microplate luminometer (Promega Corporation).

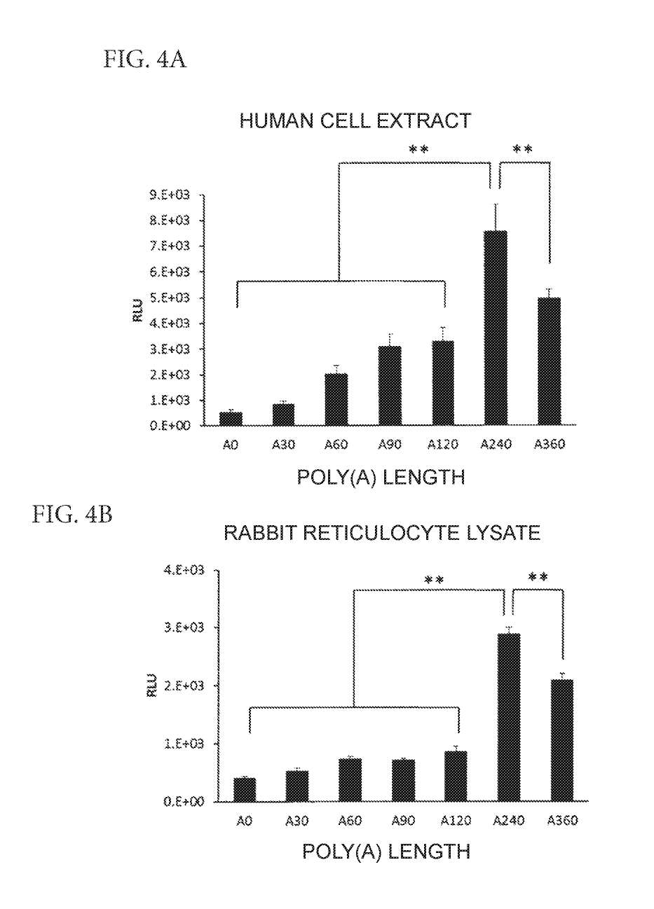

The results were as shown in FIG. 4. As shown in FIGS. 4A and 4B, the protein expression level of the mRNA having A(240) was significantly higher than those of the mRNAs having A(120) or A(360). It was therefore confirmed that even if a cell-free extract system is used, the expression level of an mRNA including poly(A) having a length of 240 nucleotides is significantly high compared with mRNAs including poly(A) having the other lengths.

### Example 4: Efficiency of In Vivo Translation from mRNA into Protein

In this example, the expression efficiency of luciferase protein in mouse skeletal muscle was investigated.

NLuc mRNAs having A(120), A(240), or A(360) prepared in Example 1 were used. Administration was performed using a nano-micelle type carrier (see PLoS One 8(2): e56220, 2013) containing an mRNA. Specifically, the block copolymer used for constituting the nano-micelle was PEG-PAsp(DET) including a PEG block portion having a number-average molecular weight of 12000 and an Asp(DET) block portion having a number-average degree of polymerization of 65. The PEG-PAsp(DET) is a copolymer of a poly(ethylene glycol) block and a polyaspartic acid derivative block. The aspartic acid derivative is aspartic acid of which the carboxyl group is substituted with a diethyltriamine group (—NH—CH₂—CH₂—NH—CH₂—CH₂—NH₂). The PEG-PAsp(DET) and the mRNA were respectively dissolved in a 10 mM HEPES buffer, and the both were mixed to prepare a nano-micelle solution. The nano-micelle solution was prepared such that the molar ratio, N/P ratio, of the amino group (N) of the PEG-PAsp(DET) to the phosphate group (P) of the mRNA was 8.

The micelle was administered into the lower limb skeletal muscle of a mouse by hydrodynamics method. Specifically, the mouse was anesthetized with 3% isoflurane (Abbott Japan Co., Ltd.), and a tourniquet was then indwelled at the proximal femur to temporarily block the blood circulation in the lower limb. Subsequently, 300 μL of the nano-micelle solution containing 5 μg of an mRNA was administered from the great saphenous vein behind the ankle medial malleolus over 5 seconds. After 5 minutes from the administration, the tourniquet was removed.

Subsequently, the amount of the luciferase protein expressed in the skeletal muscle after 72 hours from the administration was quantitatively measured. Specifically, the lower limb skeletal muscle after 72 hours from the administration was collected, and the tissue was homogenized with Multi-beads shocker (Yasui Kikai Corporation). The amount of the luciferase protein was quantitatively measured based on the amount of luciferase luminescence. The quantitative measurement of the amount of luminescence was carried out using Nano-GLo Luciferase assay system (Promega Corporation) and Lumat LB9507 luminometer (Berthold Technologies), and the amount of luminescence was standardized by the protein concentration in the cell lysate.

As a result, as shown in FIG. 4, the mRNA including A(240) showed protein expression at a significantly high level compared with the mRNA including A(120) or A(360).

These results revealed that in order to enhance the translation efficiency of an mRNA, it is preferable to adjust the length of poly(A) to about 240 nucleotides.

### Example 5: Treatment of Sciatic Nerve Damage Model Mouse

In this example, IGF-1-expressing mRNA molecules including poly(A) having different lengths were administered to sciatic nerve damage model mice to verify the therapeutic effect.

The sciatic nerve of the left leg of each mouse (Balb/c albino mouse, female, 10 to 14 week-old, purchased from Charles River Laboratories) was exposed near the greater trochanter, and the exposed sciatic nerve was pressed with tweezers cooled with liquid nitrogen to prepare a sciatic nerve damage model mouse.

Poly (A) having a length of 120 nucleotides or 240 nucleotides was added to the 3′UTR region of an IGF-1-expressing mRNA, and the mRNA was mixed with a PEG-poly(N′—[N-(2-aminoethyl)-2-aminoethyl]-aspartic acid) block copolymer (PEG-PAsp(DET)) to form a polyion complex micelle.

The PEG-PAsp(DET) was prepared according to an ordinary method (see ChemMedChem, 1 (2006), 439-444). It was estimated by H¹-NMR measurement that the PEG portion had a number-average molecular weight of 12000 and the PAsp(DET) portion had a degree of polymerization of 69. The resulting PEG-PAsp(DET) block copolymer and the mRNA were each dissolved in 10 mM Tris-HCl (pH 7.4), and the resulting solutions were mixed with each other to form a polyion complex micelle for administration.

Avascularization was performed near the femur of the mouse, and the resulting micelle was intravenously administered to the leg of the disease model to allow the micelle to permeate into the muscular tissue. In order verify the therapeutic effect, the motor function of the mouse was evaluated for the period from the 7th to the 28th day after the administration. As a negative control, the motor function of a sciatic nerve damage model mouse administered with an mRNA for luciferase, instead of IGF-1, was evaluated.

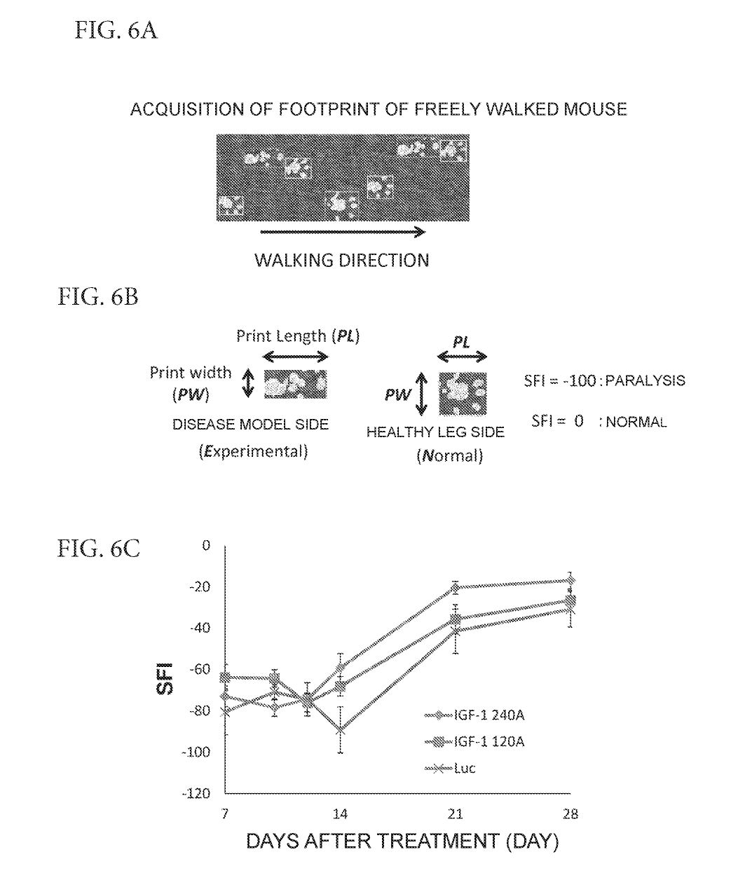

The evaluation of the motor function was performed by obtaining footprints of a freely walked mouse as shown in FIG. 6A and using the Sciatic Functional Index (SFI), which is known as an index for evaluating recovery of motor function, as an index (regarding the SFI, see Inserra M M, et al., Microsurgery, 1998, 18(2): 119-124). The SFI was calculated based on the following expression:

$$SFI = - 51.2 ( \frac{EPL - NPL}{NPL} ) + 118.9 ( \frac{EPW - NPW}{NPW} ) - 7.5 \qquad [ Expression 1 ]$$

{where, EPL represents the length in the walking direction of the footprint of the leg on the disease model side; NPL represents the length in the walking direction of the footprint of the healthy leg; the EPW represents the width of the footprint of the leg on the disease model side; and NPW represents the width of the footprint of the healthy leg}.

In the acquisition of the footprints, a walking analyzer CatWalk (manufactured by Noldus Inc.) was used. EPL, NPL, EPW, and NPW are the same as those described in the above-mentioned expression and are also shown in FIG. 6B. Ideally, SFI is −100 when a leg is completely paralyzed and is 0 when a leg is normal, and an increase in the SFI value means recovery of motor function of the leg.

An IGF-1-expressing mRNA was administered to the leg on the disease model side of the sciatic nerve damage model mouse, and the walking recovery of the mouse on the 7th, 10th, 12th, 14th, 21st, and 28th days after the administration was investigated using the SFI value as the index. The results were as shown in FIG. 6C.

As shown in FIG. 6C, although the motor function recovered with time even in the negative control, significant recovery of the motor function was observed in the case of the poly(A) having a length of 240 (IGF-1 240A). In addition, considerable recovery of the motor function was observed in the case of the poly(A) having a length of 240 (IGF-1 240A), compared to the case of the poly(A) having a length of 120 (IGF-1 120A).

Thus, the expression level of the protein from an mRNA is significantly increased by adjusting the poly(A) of the mRNA to a certain length (in particular, a length of 240 nucleotides), and this effect was confirmed in vivo.

1. A composition, comprising:
an mRNA encoding a target protein, wherein
80% or more of mRNA molecules contained in the composition have a sequence consisting substantially of poly(A) having a length of 230 to 250 nucleotides on a 3′-end side of a protein-coding region thereof.

2. The composition according to claim 1, wherein 90% or more of the mRNA molecules contained in the composition have a sequence consisting substantially of poly(A) having a length of 230 to 250 nucleotides on the 3′-end side of the protein-coding region thereof.

3. The composition according to claim 1, wherein 95% or more of the mRNA molecules contained in the composition have a sequence consisting substantially of poly(A) having a length of 230 to 250 nucleotides on the 3′-end side of the protein-coding region thereof.

4. The composition according to claim 1, wherein 20% or less of the mRNA molecules contained in the composition have poly(A) having a length of 270 or more nucleotides.

5. A protein expression vector, comprising:
a gene encoding a protein and operably linked to a promoter; and
a sequence consisting substantially of poly(A) having a length of 230 to 250 nucleotides downstream of a protein-coding region of the gene encoding the protein.

6. A method for improving an ability of an mRNA to bind to eIF4E, the method comprising:
adding a sequence consisting of poly(A) or consisting substantially of poly(A) to a downstream side of a protein-coding region of a DNA to be transcribed into the mRNA such that the mRNA has a sequence consisting substantially of poly(A) having a length of 230 to 250 nucleotides on a 3′-end side of the protein-coding region thereof.

7. A method for expressing a target protein in a cell in a body of a subject, comprising:
providing a composition comprising an mRNA encoding the target protein, where 80% or more of mRNA molecules contained in the composition have a sequence consisting substantially of a poly(A) sequence having a length of 230 to 250 nucleotides on a 3′-end side of a protein-coding region of the mRNA; and
administering the composition to the subject.

8. A pharmaceutical composition, comprising:
an mRNA encoding a protein that can treat a disease or a disorder in a subject suffering from the disease or a subject having the disorder, wherein 80% or more of mRNA molecules contained in the composition have a sequence consisting substantially of a poly(A) sequence having a length of 230 to 250 nucleotides on a end side of a protein-coding region of the mRNA.

9. The composition according to claim 8, wherein the disease or the disorder is a disease or a disorder caused by a reduction or a lack of a protein, and the protein that can treat the disease or the disorder is the protein that is reduced or lacked in the subject.

10. The composition according to claim 8, wherein the disease or the disorder is spinal cord injury, and the protein that can treat the disease or the disorder is brain-derived neurotrophic factor (BDNF).

11. The composition according to claim 8, wherein the disease or the disorder is peripheral nerve injury, and the protein that can treat the disease or the disorder is insulin-like growth factor (IGF-1).


SEQUENCE LISTING (25 sequences)

SEQ ID NO: 1 - DNA, 3048 bp, Artificial Sequence
  pSP73-Luc
gaaccagatctgatatcatcgatgaattcgagctcggtacccggggatcctctagatgcatgctcgagcggccgcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccatgggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccatctctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatggtggctggatccgagctcggtaccaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 2 - DNA, 133 bp, Artificial Sequence
  5′-AATTC-A121-GAGACGA-3′
aattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagacga

SEQ ID NO: 3 - DNA, 133 bp, Artificial Sequence
  5′-GATCTCGTCTC-T121-G-3′
gatctcgtctctttttttttttttttttttttttttttttttttttttttttttttttttttttttttttttttttttttttttttttttttttttttttttttttttttttttttttttttttttttttttg

SEQ ID NO: 4 - DNA, 3163 bp, Artificial Sequence
  pSP73-Luc+T121
gaaccagatctcgtctctttttttttttttttttttttttttttttttttttttttttttttttttttttttttttttttttttttttttttttttttttttttttttttttttttttttttttttttttttttttttgaattcgagctcggtacccggggatcctctagatgcatgctcgagcggccgcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccatgggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccatctctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatggtggctggatccgagctcggtaccaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 5 - DNA, 132 bp, Artificial Sequence
  5′-AATTC-A120-GATATCA-3′
aattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagatatca

SEQ ID NO: 6 - DNA, 132 bp, Artificial Sequence
  5′-GATCTGATATC-T120-G-3′
gatctgatatcttttttttttttttttttttttttttttttttttttttttttttttttttttttttttttttttttttttttttttttttttttttttttttttttttttttttttttttttttttttttg

SEQ ID NO: 7 - DNA, 3162 bp, Artificial Sequence
  pSP73-GLuc-A(120)-EcoRV
gaaccagatctgatatcttttttttttttttttttttttttttttttttttttttttttttttttttttttttttttttttttttttttttttttttttttttttttttttttttttttttttttttttttttttttgaattcgagctcggtacccggggatcctctagatgcatgctcgagcggccgcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccatgggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccatctctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatggtggctggatccgagctcggtaccaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 8 - DNA, 69 bp, Artificial Sequence
  5′-G-A61-GAGACGA-3′
gaaaaaaaaaaaaaaaaaaaaaaaaaaaaaaaaaaaaaaaaaaaaaaaaaaaaaaaaaaaaagagacga

SEQ ID NO: 9 - DNA, 73 bp, Artificial Sequence
  5′-GATCTCGTCTC-T61-C-3
gatctcgtctctttttttttttttttttttttttttttttttttttttttttttttttttttttttttttttc

SEQ ID NO: 10 - DNA, 3227 bp, Artificial Sequence
  pSP73-GLuc-A(120)-EcoRV+T61
gaaccagatctcgtctctttttttttttttttttttttttttttttttttttttttttttttttttttttttttttttcatcttttttttttttttttttttttttttttttttttttttttttttttttttttttttttttttttttttttttttttttttttttttttttttttttttttttttttttttttttttttttgaattcgagctcggtacccggggatcctctagatgcatgctcgagcggccgcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccatgggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccatctctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatggtggctggatccgagctcggtaccaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 11 - DNA, 99 bp, Artificial Sequence
  5′-G-A91-GAGACGA-3′
gaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagacga

SEQ ID NO: 12 - DNA, 103 bp, Artificial Sequence
  5′-GATCTCGTCTC-T91-C-3
gatctcgtctctttttttttttttttttttttttttttttttttttttttttttttttttttttttttttttttttttttttttttttttttttttttttttc

SEQ ID NO: 13 - DNA, 3257 bp, Artificial Sequence
  pSP73-GLuc-A(120)-EcoRV+T91
gaaccagatctcgtctctttttttttttttttttttttttttttttttttttttttttttttttttttttttttttttttttttttttttttttttttttttttttttcatcttttttttttttttttttttttttttttttttttttttttttttttttttttttttttttttttttttttttttttttttttttttttttttttttttttttttttttttttttttttttgaattcgagctcggtacccggggatcctctagatgcatgctcgagcggccgcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccatgggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccatctctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatggtggctggatccgagctcggtaccaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 14 - DNA, 129 bp, Artificial Sequence
  5′-G-A121-GAGACGA-3′
gaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagacga

SEQ ID NO: 15 - DNA, 133 bp, Artificial Sequence
  5′-GATCTCGTCTC-T121-C-3
gatctcgtctctttttttttttttttttttttttttttttttttttttttttttttttttttttttttttttttttttttttttttttttttttttttttttttttttttttttttttttttttttttttttc

SEQ ID NO: 16 - DNA, 3287 bp, Artificial Sequence
  pSP73-GLuc-A(120)-EcoRV+T121
gaaccagatctcgtctctttttttttttttttttttttttttttttttttttttttttttttttttttttttttttttttttttttttttttttttttttttttttttttttttttttttttttttttttttttttttcatcttttttttttttttttttttttttttttttttttttttttttttttttttttttttttttttttttttttttttttttttttttttttttttttttttttttttttttttttttttttttgaattcgagctcggtacccggggatcctctagatgcatgctcgagcggccgcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccatgggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccatctctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatggtggctggatccgagctcggtaccaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 17 - DNA, 128 bp, Artificial Sequence
  5′-G-A120-GATATCA-3′
gaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagatatca

SEQ ID NO: 18 - DNA, 132 bp, Artificial Sequence
  5′-GATCTGATATC-T120-C-3′
gatctgatatcttttttttttttttttttttttttttttttttttttttttttttttttttttttttttttttttttttttttttttttttttttttttttttttttttttttttttttttttttttttttc

SEQ ID NO: 19 - DNA, 3286 bp, Artificial Sequence
  pSP73-GLuc-A(240)-EcoRV
gaaccagatctgatatcttttttttttttttttttttttttttttttttttttttttttttttttttttttttttttttttttttttttttttttttttttttttttttttttttttttttttttttttttttttttcatcttttttttttttttttttttttttttttttttttttttttttttttttttttttttttttttttttttttttttttttttttttttttttttttttttttttttttttttttttttttttgaattcgagctcggtacccggggatcctctagatgcatgctcgagcggccgcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccatgggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccatctctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatggtggctggatccgagctcggtaccaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 20 - DNA, 39 bp, Artificial Sequence
  5′-G-A31-GAGACGA-3′
gaaaaaaaaaaaaaaaaaaaaaaaaaaaaaaagagacga

SEQ ID NO: 21 - DNA, 43 bp, Artificial Sequence
  5′-GATCTCGTCTC-T31-C-3′
gatctcgtctctttttttttttttttttttttttttttttttc

SEQ ID NO: 22 - DNA, 3321 bp, Artificial Sequence
  pSP73-GLuc-A(240)-EcoRV+T31
gaaccagatctcgtctctttttttttttttttttttttttttttttttcatcttttttttttttttttttttttttttttttttttttttttttttttttttttttttttttttttttttttttttttttttttttttttttttttttttttttttttttttttttttttttcatcttttttttttttttttttttttttttttttttttttttttttttttttttttttttttttttttttttttttttttttttttttttttttttttttttttttttttttttttttttttttgaattcgagctcggtacccggggatcctctagatgcatgctcgagcggccgcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccatgggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccatctctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatggtggctggatccgagctcggtaccaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 23 - DNA, 3411 bp, Artificial Sequence
  pSP73-GLuc-A(240)-EcoRV+T121
gaaccagatctcgtctctttttttttttttttttttttttttttttttttttttttttttttttttttttttttttttttttttttttttttttttttttttttttttttttttttttttttttttttttttttttttcatcttttttttttttttttttttttttttttttttttttttttttttttttttttttttttttttttttttttttttttttttttttttttttttttttttttttttttttttttttttttttcatcttttttttttttttttttttttttttttttttttttttttttttttttttttttttttttttttttttttttttttttttttttttttttttttttttttttttttttttttttttttttgaattcgagctcggtacccggggatcctctagatgcatgctcgagcggccgcttagtcaccaccggcccccttgatcttgtccacctggccctggatcttgctggcaaaggtcgcacagcgttgcggcagccacttcttgagcaggtcagaacactgcacgttggcaagccctttgaggcagccagttgtgcagtccacacacagatcgacctgtgcgatgaactgctccatgggctccaagtccttgaacccaggaatctcaggaatgtcgacgatcgcctcgcctatgccgccctgtgcggactctttgtcgccttcgtaggtgtggcagcgtcctgggatgaacttcttcatcttgggcgtgcacttgatgtgggacaggcagatcagacagcccctggtgcagccagctttccgggcattggcttccatctctttgagcacctccagcggcagcttcttgccgggcaacttcccgcggtcagcatcgagatccgtggtcgcgaagttgctggccacggccacgatgttgaagtcttcgttgttctcggtgggcttggcctcggccacagcgatgcagatcagggcaaacagaactttgactcccatggtggctggatccgagctcggtaccaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 24 - DNA, 3309 bp, Artificial Sequence
  IGF-1 240A
gaaccagatctcgtctctttttttttttttttttttttttttttttttttttttttttttttttttttttttttttttttttttttttttttttttttttttttttttttttttttttttttttttttttttttttttcatcttttttttttttttttttttttttttttttttttttttttttttttttttttttttttttttttttttttttttttttttttttttttttttttttttttttttttttttttttttttttgaattcgagctcggtacccttgaacaaataacgtcgaatattaccaatgtttatttcgttatcgtagtgtttaaagtgtttatttcgttatcgtagtgtttaaagtgtttatttcgtaaaaaaagtgacgtaagatcaacaccaaacaggtttgagtagttacatagaatagtacagctcgatcgccactacaccgtaaaagacgaggcaccctccgaggaggatgtaagacatccagaacaaaggacgtgaaggagatgaacacaagaagtttacatgaaggaagactcagaacccgtacagtcacaccgcgacccgtgcctatctcgcccgacgaaaacatccgaagtcaccccgtgtcatgtagaggtcagaggagtctagtgtcgaggccttcgttgtgagtaggtgttacggacagactccacgggaggcttacgacctcggtatcggacacccgaacaacttcattttcggggagccaggtgtgtgcttgacttctcgtaggtggtcgagtcggggcgtttcccagagaccaggtcgacaccacctcgaccacttccactcgttcgtctcgcggtccatcttctccacacttctgctgtacaacacggaggagtcgacttggccactagagtcgacatcctttctcttcttccaatcatcagcttcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 25 - DNA, 3184 bp, Artificial Sequence
  IGF-1 120A
gaaccagatctcgtctcttttttttttttttttttttttttttttttttttttttttttttttttttttttttttttttttttttttttttttttttttttttttttttttttttttttttttttttttttttttttgaattcgagctcggtacccttgaacaaataacgtcgaatattaccaatgtttatttcgttatcgtagtgtttaaagtgtttatttcgttatcgtagtgtttaaagtgtttatttcgtaaaaaaagtgacgtaagatcaacaccaaacaggtttgagtagttacatagaatagtacagctcgatcgccactacaccgtaaaagacgaggcaccctccgaggaggatgtaagacatccagaacaaaggacgtgaaggagatgaacacaagaagtttacatgaaggaagactcagaacccgtacagtcacaccgcgacccgtgcctatctcgcccgacgaaaacatccgaagtcaccccgtgtcatgtagaggtcagaggagtctagtgtcgaggccttcgttgtgagtaggtgttacggacagactccacgggaggcttacgacctcggtatcggacacccgaacaacttcattttcggggagccaggtgtgtgcttgacttctcgtaggtggtcgagtcggggcgtttcccagagaccaggtcgacaccacctcgaccacttccactcgttcgtctcgcggtccatcttctccacacttctgctgtacaacacggaggagtcgacttggccactagagtcgacatcctttctcttcttccaatcatcagcttcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata